(19) United States
(12) Patent Application Publication
(10) Pub. No.: US 2002/0197683 A1
(43) Pub. Date: Dec. 26, 2002

(54) Metabolic controlled fermentation process for carbamoyl tobramycin production

(76) Inventors: Istvan Bakondi-Kovacs, Karcag (HU); Ilona Csutoros Novotny, Budapest (HU); Janos Erdei, Debrecen (HU); Gabor Balogh, Debrecen (HU); Peter Seress, Debrecen (HU)

Correspondence Address: KENYON & KENYON, ONE BROADWAY, NEW YORK, NY, 10004, US

(21) Appl. No.: 10/047,693

(22) Filed: Jan. 9, 2002

Related U.S. Application Data
(60) Provisional application No. 60/260,542, filed on Jan. 9, 2001.
(60) Provisional application No. 60/337,127, filed on Dec. 4, 2001.

(51) Int. Cl. C12P 19/26

(52) U.S. Cl. 435/84

(57) ABSTRACT

A metabolic controlled fermentation process has been developed for the production of carbamoyl tobramycin by the application of different Streptomyces tenebrarius strains in submerged cultures at a temperature within about 37-41° C. on a medium containing assimilable carbon and nitrogen sources, mineral salts and controlling the assimilable carbon and nitrogen sources by feeding in an optimal range. As a result of this invention a high yield production of carbamoyl tobramycin with high purity could be achieved.

### CROSS-REFERENCE TO RELATED APPLICATIONS

This application claims the benefit under 35 U.S.C. §119(e) of the U.S. Provisional Patent Application Ser. Nos. 60/260,542 filed Jan. 9, 2001 and the U.S. Provisional Patent Application No. 60/337,127 filed Dec. 4, 2001 entitled “Metabolic Controlled Fermentation Process for Carbamoyl Tobramycin Production” by Estavan BAKONDI-KOVACS, Ilona Csutoros NOVOTNY, Janos ERDEI, Gabor BALOGH, Peter SERESS; the content of which is incorporated herein by reference.

### FIELD OF THE INVENTION

The present invention relates to the development of a metabolic controlled fermentation process for 6′-0-carbamoyl tobramycin production.

More specifically, the invention discloses cultivation of Streptomyces tenebrarius strains to produce 6′-0-carbamoyl tobramycin by controlling the fermentation process through regulating the levels of glucose, glutamic acid and ammonia nitrogen.

### BACKGROUND OF THE INVENTION

Tobramycin has the chemical name O-3-amino-3-deoxy-α-D-glucopyranosyl-(1→6)-O-[2,6-diamino-2,3,6-trideoxy-α-D-ribo-hexo-pyranosyl-(1→4)]-2-deoxy-D-streptamine [a/k/s “4-[2,6-diamino-2,-3 ,6-trideoxy-α-D-glycopyranosyl]-6-[3-amino-3-deoxy-α-D-glycopranosyl]-2-deoxystreptamine”, nebramycin factor 6; NF 6; Gemebcin; Tobracin; Tobradistin; Tobralex; Tobramaxin; Tobrex. Tobramycin has the chemical formula of:

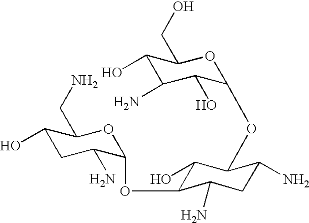

Tobramycin is an antibiotic that has a broad spectrum of activity against both Gram positive and Gram negative bacteria. Sensitive bacteria include Staphylococcus aureus, Staphylococcs epidermidis, Streptococcus pneumoniae, Psudomonas aeruginosa, Escherichia coli, Enterobacter aerogenes, Proteus mirabelis, Klebsiella pneumoniae, Morganella morganii, Haemophilus influenzae, Haemophilus aegyptius, Moraxlea lacumata, and Acinetobacter calcoaceticus. Tobramycin is known to have a good anti-bacterial profile in eye and ear infections.

Tobramycin is presently produced by the cultivation of Streptomyces tenebrarius. Fed batch technology is often used in the production of carbamoyl tobramcyin. hi batch fermentation, the metabolism of carbon and nitrogen is not controlled directly. Due to the depletion of nutrients, which occurs during the cultivation period, the yield of carbamoyl tobramycin is substantially reduced. Carbamoyl tobramycin fermentation is also notably sensitive to oxygen supply. Additionally, volume loss resulting from evaporation during cultivation also affects the yield and volume compensation during the cultivation introduces a risk of contamination.

It is desirable to develop a technology whereby the fine correction of feeding profiles in the course of fermentation can be regulated by a fine-controlled technology to improve fermentation production for carbamoyl tobramycin with substantially higher yield and purity.

### OBJECTS OF THE INVENTION

It is therefore an object of the present invention to provide an economical and high efficient process for producing carbamoyl tobramycin. The disclosed process involves cultivation of 6′-0-carbamoyl tobramycin producing microorganisms and relates to the metabolic control of the fermentation process of 6′-0-carbamoyl tobramycin by such microorganisms so as to produce a substantially increased purity.

It is a further object of the present invention to selectively regulate a constant level of nutrition during the cultivation of 6′-0-carbamoyl tobramycin producing microorganisms.

It is yet another object of the present invention to provide for metabolic control of the fermentation process of 6′-0-carbamoyl tobramycin by independently maintaining the levels of glucose, glutamic acid and ammonia nitrogen.

### SUMMARY OF THE INVENTION

The present invention provides a high yield fermentation process for the production of 6′-0-carbamoyl tobramycin in submerged cultures at a temperature in the range of about 37° C. to about 41° C. in a medium comprising assimilable carbon and nitrogen sources and a mineral salt.

The process preferably includes the steps of cultivating a 6′-0-tobramycin producing strain of microorganism in a fermentation broth stable for the production of 6′-0-carbamoyl tobramycin, whereby the carbon and nitrogen metabolism of the strain during the secunder metabolism are controlled at a glucose level of about 0.001 to about 0.5%, glutamic acid level of about 0.005 to about 0.1% and ammonia nitrogen level of about 0.03 to about 0.2% by feeding continuously the glucose, sodium glutamate and ammonium solution. In the method according to the invention, the regulation of nutrient is preferably conducted independently of each other.

According to one embodiment, the inorganic phosphate is fed during the fermentation in a quantity of about 0.001 to about 0.002% per day.

The present invention provides a process for producing 6′-0-carbamoyl tobramycin from Streptomyces tenebrarius while metabolically controlling the production of 6′-0-carbamoyl tobramycin, comprising the steps of: a) preparing a fermentation broth containing the 6′-0-carbamoyl tobramycin producing microorganism; b) regulating a constant level of assimilable carbon source and assimilable nitrogen source; and c) recovering the 6′-0-carbamoyl tobramycin.

The present invention provides that the fermentation medium has a temperature range of about 37° C. to about 41° C.

The present invention provides that the fermentation medium is a submerged culture.

The present invention provides that the fermentation broth contains assimilable carbon, assimilable nitrogen sources, mineral salts using different Streptomyces tenebrarius strains.

The present invention provides that the assimilable carbon and nitrogen sources are controlled at a glucose level of about 0.001 to about 0.5%. The present invention further provides that the assimilable carbon and nitrogen sources are controlled at a glutamic acid level of about 0.005 to about 0.1%. The present invention further provides that the assimilable carbon and nitrogen sources are controlled at an ammonia nitrogen level of about 0.03 to about 0.2%.

The present invention provides that the assimilable carbon and nitrogen sources are controlled by feeding continuously a glucose, sodium glutamate and ammonium (NH₄⁺) solution independently of each other.

The present invention further provides for adjusting the glucose pH with phosphoric acid. Preferably the pH range of the glucose solution is about 4 to about 5. The invention also provides an inorganic phosphate may be fed to the fermentation medium with glucose is in a quantity of about 0.001 to about 0.002% per day.

### DETAILED DESCRIPTION OF THE INVENTION

As used herein, the terms “ppm” refers to parts per million; “rpm” refers to revolutions per minute, and “vvm” refers to volume per volume per minute.

As used herein, the term “NH₃-N” refers to ammonia nitrogen.

Unless otherwise specified, the term “%” refers to % weight vs. weight. For example, 0.001% glucose means 0.001 gram glucose vs. 100 gram of the fermentation broth.

As used herein, the term “6′-O-carbomoyl-tobramycin” refers to a carbamoylised form of tobramycin. During the synthesis of tobramycin, tobramycin is biosynthesized in a carbamoylised form which is the 6′-O-carbomoyl-tobramycin. It is also known as carbamoyl tobramycin.

As used herein, the term “fed batch technology” refers to a fermentation where one or more nutrient components added to the batch during the fermentation process. When one or two increments of nutrient is added during fermentation (about 1 to about 2%), it is called the bang-bang fermentation. When a large number of small portion of nutrient is added during fermentation (about 0.02 to about 0.05%) or true (uninterrupted) continuous feeding, it is called the continuous feeding fermentation. As used herein, the term “continuous feed” refers to small portion feeding (about 0.02% to about 0.05%) or truly continuous feeding or nutrients and oxygen.

As used herein, the term “assimilable” refers to a given microorganism that has an enzyme system for absorption of nutrients and consumption or use or decompose of such nutrients to use in the biosynthesis of complex constituents of the microorganism.

As used herein, the term “a mineral salt” refers to a salt of biologically important element and trace element which includes calcium, magnesium, iron, zinc, phosphate, manganese, sodium, potassium, and cobalt.

As used herein, the term “main fermenter” refers to a vessel used in the fermentation process used for growing of Streptomyces and for the production of 6′-O-carbamoyl tobramycin.

Accordingly, the invention provides a process for producing 6′-O-carbamoyl tobramycin by individually control the fermentation process; preferably, by continuously regulating the levels of glucose, glutamic acid and ammonia nitrogen; most preferably each independently of the other.

According to the present invention, tobramycin is biosynthesized in a carbamoylised form, that is, the 6′-O-carbomoyl-tobramycin. The type, rate, and ratio of carbon and nitrogen metabolism is important in the formation of 6′-O-carbamoyl tobramycin. In batch fermentation, this metabolism is not controlled directly. The present invention provides for optimizing glucose and glutamic acid levels in a fermentation broth, and optimizing ratio of the carbon o nitrogen metabolism for the forming carbamoyl tobramycin. Based on this information, the present invention further provides a new fermentation technology for the production of 6′-O-carbamoyl tobramycin (i.e., controlled fed batch technology). While fed batch technologies for other fermentation products are generally well-known and used; the present invention provides a controlled fed batch technology for 6′-O-carbamoyl tobramycin by controlling the metabolism of assimilable carbon and nitrogen that is unique for 6′-O-carbamoyl tobramycin.

In the fermentation process of this invention, different assimilable carbon and nitrogen sources can be used. A preferred embodiment of the present invention involves using glucose or glutamic acid (or its salt) as a assimilable carbon source. Another preferred embodiment of the present invention involves using ammonia nitrogen as a assimilable nitrogen source.

According to the present invention, the assimilable nitrogen source is selected from the group of metabolizable organic and inorganic compounds. Such compounds include urea, ammonium sulfate, ammonium chloride, ammonium phosphate, ammonium nitrate and the like, and mixtures thereof. Preferably, ammonia nitrogen is ammonium sulfate [(NH₄)₂SO4].

According to the present invention, regulating the levels of glucose, glutamic acid and “ammonia nitrogen” is important in the biosynthesis of carbamoyl tobramycin. The present invention provides a fermentation process for 6′-O-carbamoyl tobramycin where “at least one of the levels of glucose, glutamic acid or ammonia nitrogen” is controlled or regulated at a constant level, resulting a better yield and purity of 6′-O-carbamoyl tobramycin.

A preferred 6′-O-carbamoyl tobramycin producing microorganism for carrying out the fermentation process of the invention is Streptomyces tenebrarius. Preferably the Streptomyces tenebrarius is the Streptomyces tenebrarius strain deposited as NCAIM B(P) 000169. Preferably the Streptomyces tenebrarius is the Streptomyces tenebrarius strain deposited as NCAIM B(P) 000204.

In one embodiment of the invention, the glucose level is regulated at about 0.001 to about 0.5%. Preferably, the glucose level is regulated at about 0.001 to about 0.4%. Most preferably, the glucose level is regulated at about 0.001 to about 0.05%.

In another embodiment of the invention, the glutamic acid level is regulated at about 0.005 to about 0.1%. More preferably, the glutamic acid level is regulated at about 0.001 to about 0.1%.

In another preferred embodiment of the invention, the glutamic acid in the salt from (e.g., sodium glutamate) is regulated at about 0.005 to about 0. 1%. More preferably, the glutamate level is regulated at about 0.001 to about 0.1%.

In another embodiment of the invention, the ammonia nitrogen level is regulated at about 0.03 to about 0.2%. More preferably, the ammonia nitrogen level is regulated at about 0.02 to 0.2%.

Preferably, the metabolic controlled fermentation of 6′-O-carbamoyl tobramycin is conducted by feeding continuously the glucose, sodium glutamate and ammonia nitrogen solution independently of each other.

Tobramycin is an aminoglycoside type antibiotic. During its biosynthesis of 6′-O-carbamoyl tobramycin, there are two ways of glucose catabolism: Embden-Mayerhoff-Pamass cycle and Hexose-Monophosphate shunt in which catabolic products may repress the 6′-O-carbamoyl tobramycin biosynthesis. The metabolic controlled fermentation is regulated by maintaining the level of glucose in the fermentation broth. Preferably, the glucose is maintained at a low level (e.g., about 0.001 to about 0.5%) to assure the absence of glucose catabolites (or glucose-catabolite intermediates) repression.

Similarly, the metabolic controlled fermentation is regulated by maintaining the level of glutamic acid in the fermentation broth. Preferably, the glutamic acid (or its salt) is maintained at a low level (e.g., about 0.005 to about 0.1%) to assure the absence of gluatmate catabolites repression.

Similarly, the metabolic controlled fermentation is regulated by maintaining the level of ammonia nitrogen in the fermentation broth. Preferably, the ammonia nitrogen is maintained at a low level (e.g., about 0.03 to about 0.2%). Regulating the ammonia nitrogen level at a low level assures the ample supply of substrates for the transamination process without the problems associated with the catabolic products.

The present invention provides the metabolic controlled fermentation by maintaining the level of at least one of glucose, glutamic acid and ammonia nitrogen.

In another embodiment of the invention, inorganic phosphate is fed into the fermentation medium with the proviso that the overall amount thereof is sufficient to permit the fermentation process to proceed effectively. Preferably the quantity of the inorganic phosphate is in the quantity range of about 0.001 to about 0.002% per day.

The present invention provides the metabolic controlled fermentation of 6′-O-carbamoyl tobramycin wherein the improved yield of 6′-O-carbamoyl tobramycin is generally greater than about 30%.

The invention is further described in the following examples which are in no way intended to limit the scope of the invention.

### EXAMPLES

### Example 1

| | Seed medium, | Main fermentation |
|---|---|---|
| | gram/liter | medium, gram/liter |
| Dextrose monohydrate | 30 | 50 |
| Soya bean meal | 20 | 35 |
| Acidic casein | 2.5 | 6.75 |
| Pancreatin | 0.05 | 0.17 |
| Ammonium chloride | 3 | 5 |
| Ammonium nitrate | 1 | — |
| Magnesium sulphate | 5 | — |
| Cobalt nitrate | 0.01 | 0.01 |
| Calcium carbonate | 3 | 5 |
| Soya bean oil | 15 | 16 |
| Palm oil | 15 | 16 |
| Zinc sulphate | — | 1 |

### Culture of Streptomyces tenebrarius in Seed Medium

A seed medium (without glucose) was prepared in a 60 liter vessel. The seed medium was sterilised at about 121° C. for about 60 min.

A glucose solution was separately prepared. The pH of the glucose solution was adjusted by hydrochloric acid to about 4.0 to about 5.0 value. Sterilisation of the glucose solution was done at about 121° C. for about 30 min. The sterilised glucose solution was added into the sterilised seed medium.

The Streptomyces tenebrarius strain (NCAIM B(P) 000169) was inoculated in a quantity of about 500 ml of the sterile seed medium (with glucose). A vegetative cell culture of Streptomyces tenebrarius strain was allowed to grow to a logarithmic phase. Cultivation was carried out at the parameters of temperature: about 37° C., head pressure: about 0.4 bar, mixing rate: about 2.6 m/sec and aeration ration: about 0.4 ppm.

### Culture of Streptomyces tenebrarius in Fermentation Medium

A main fermentation medium (without glucose) was prepared in a 300 liter vessel. The main fermentation medium was sterilised at about 121° C. for about 60 min.

A glucose solution was separately prepared. The glucose solution was adjusted to a pH of about 4.0 to about 5.0 using hydrochloric acid. The glucose solution was sterilised at about 121° C. for about 30 min. The sterilised glucose solution was added into the main fermentation medium after sterilisation.

Transferring of the seed stage to the main fermenter was after 24 hours cultivation. The seed stage to main fermentation transferring ratio was 10%. Cultivation parameters for the main fermenter were as follows. Temperature within 0-70 hours: about 37° C. and within 70 hours-till the end of fermentation process: about 39° C.; aeration rate: about 0.1 ppm; stirring rate: about 250 rpm; internal pressure: about 0.2 bar.

A solution of sodium glutamate in a quantity of 8 gram/liter medium was prepared. A solution of magnesium sulphate in 10 gram/liter medium was also prepared. Both solutions of sodium glutamate and magnesium sulphate were sterilised at about 121° C. for about 60 min. Both solutions were then added in 20 liter volume into the main fermentation culture at its age of 24 hours. Cultivation was done for 144 hours.

Initial glucose content of the medium was exhausted by the 80^{th} hour of the fermentation. Initial glutamate content of the medium was consumed completely by the 60^{th} hour of the fermentation. Initial 120 mg/100 mL NH₃-N content (as measured by the “Formol” titration) of the medium reduced to below 60 mg/100 mL by the 50^{th} hour of the fermentation and was consumed completely by the end of the fermentation.

The achieved yield measured by HPLC was 1,856 μg/gram apramycin, 678 μg/gram carbamoyl kanamycin and 1,968 μg/gram 6′-0-carbamoyl tobramycin.

### Example 2

| | Seed medium, | Main fermentation |
|---|---|---|
| | gram/liter | medium, gram/liter |
| Dextrose monohydrate | 30 | 50 |
| Soya bean meal | 20 | 50 |
| Magnesium sulphate | 5 | |
| Ammonium sulphate | 3 | 5 |
| Calcium carbonate | 3 | 5 |
| Soya bean oil | 30 | 32 |
| Zinc sulphate | — | 1 |
| Potassium dihydrogen | — | 0.45 |
| phosphate | | |

### Culture of Streptomyces tenebrarius in Seed Medium

A seed medium was prepared in a 60 liter vessel. The seed medium was sterilised at about 121° C. for about 60 min.

A glucose solution was separated prepared. The glucose solution was adjusted using hydrochloric acid to about 4.0 to about 5.0. The glucose medium was sterilised at about 121° C. for about 30 min. The sterilised glucose medium was added into the seed medium after sterilisation.

The Streptomyces tenebrarius strain (NCAIM B(P) 000169) was inoculated into a quantity of about 500 ml of sterilised seed medium. A vegetative cell culture Streptomyces tenebrarius strain was allowed to grow to a logarithmic phase. Cultivation parameters were similar to that described in Example 1.

### Culture of Streptomyces tenebrarius in Main Fermentation Medium

A main fermentation medium was prepared in a 300 liter vessel. The main fermentation medium was sterilised at about 121° C. for about 60 min.

A glucose solution was separately prepared. The glucose solution was adjusted using hydrochloric acid to about 4.0 to about 5.0. The glucose solution was sterilised at about 121° C. for about 30 min. The sterilised glucose medium was added into the main fermentation medium after sterilisation.

Condition of transferring of the seed stage to the main fermenter was similar to that described in Example 1. Cultivation time was about 20 hours. Cultivation parameters with feeding done at the 24^{th} hour were similar to that described in Example 1.

The exhaustion (i.e., consumption) of glucose, glutamate and the ammonia nitrogen content of the medium were also similar to that described in Example 1.

The achieved yield measured by HPLC was 2,150 μg/gram 6′-0-carbamoyl tobramycin.

### Example 3

A seed culture medium was prepared similarly to that described in Example 2. Inoculation was done by 500 mL vegetative culture of the Streptomyces tenebrarius strain (NCAIM B(P) 000204). Cultivation parameters were similar to that described in Example 1.

A main fermentation medium was prepared similarly to that described in Example 2. Condition of transferring of the seed stage was similar to that described in Example 1 and the cultivation time was about 18 hours. Cultivation parameters with feeding done at the 24^{th} hour were similar to that described in Example 1.

Exhaustion of glucose, glutamate and the ammonia nitrogen content of the medium were also similar to that described in Example 1.

The achieved yield measured by HPLC was 2,210 μg/gram 6′-0-carbamoyl tobramycin.

### Example 4

A seed culture medium was prepared similarly to that described in Example 2. Inoculation was done by 500 mL vegetative culture of the Streptomyces tenebrarius strain (NCAIM B(P) 000169). Cultivation parameters were similar to that described in Example 2.

A main fermentation medium was prepared similarly to that described in Example 2, but the pH of the glucose solution was adjusted by phosphoric acid. Condition of transferring of the seed stage was similar to that described in Example 1, but the cultivation time was 18 hours. Cultivation parameters with feeding done at the 24^{th} hour were similar to that described in Example 1.

Additionally 50% sodium glutamate solution was prepared and sterilised at 121° C. for 60 min, and then 50% glucose solution was prepared and after pH adjustment to about 4.0 to about 5.0 by phosphoric acid it was sterilised at about 121° C. for about 30 min. Phosphate content of the glucose solution was in the range of about 0.05 to about 0.2%. Feeding of these solutions were carried out from the 24^{th} hour of the fermentation till the end by controlling in the production phase the glucose and glutamate content in the range of about 0.001 to about 0.05% and about 0.001 to about 0.1%, respectively. Additionally to the above concentrations, ammonia solution was also fed in order to control the ammonia nitrogen content in the range of about 30 to about 200 mg/100 mL (i.e., about 0.03 to about 0.2%).

The achieved yield measured by HPLC was 3,150 μg/gram 6′-0-carbamoyl tobramycin.

### Example 5

A seed culture medium was prepared similarly to that described in Example 2. Inoculation was done by 500 ml vegetative culture of the Streptomyces tenebrarius strain (NCAIM B(P) 000204). Cultivation parameters were similar to that described in Example 2.

Condition of transferring of the seed stage was similar to that described in Example 1 and the cultivation time was about 16 hours.

A main fermentation medium was prepared similarly to that described in Example 4. Similar to Example 4, 50% sodium glutamate solution was prepared and sterilized at 121° C. for 60 min. A 50% glucose solution was prepared and after pH adjustment to about 4.0 to about 5.0 by phosphoric acid and then was sterilized at about 121° C. for about 30 min.

Fermentation conditions with metabolic controlled feeding were similar to that described in Example 4. Feeding of these solutions were carried out from the 24^{th} hour of the fermentation till the end by controlling in the production phase the glucose and glutamate content in the range of about 0.001 to about 0.5% and about 0.001 to about 0.1%, respectively. Additionally to the above concentration, ammonia solution was also fed in order to control the ammonia nitrogen content of the fermentation culture in the range of about 20 to about 200 mg/100 mL (i.e., about 0.02% to about 0.2%).

The achieved yield measured by HPLC was 4,030 μg/gram 6′-0-carbamoyl tobramycin.

Application of the fed-batch technology provides higher 6′-O-carbamoyl tobramycin activity in the fermentation broth.

As a result of the feeding not only the volume loss due to the evaporation is compensated, but an increasing in the working volume of the batch can be achieved as well, which results a more efficient utilisation of the fermenter volume and higher quantity of harvested active ingredient too.

Due to the possibility of fine correction of feeding profiles in the course of the fermentation a sophisticated, high-level controlled technology can be obtained. The present invention provides a fermentation process whereby a fine correction of feeding profiles is ensured because the levels of glucose, glutamate, and ammonia nitrogen are regulated.

Carbamoyl tobramycin fermentation is very sensitive to the oxygen supply. This parameter can be controlled more easily in the case of the fed-batch technology via adjusting the internal pressure and aeration rate to the optimally demanded value. For instance, using an aeration rate higher than 0.lvvm or a back-pressure high than 0.2 bar, the 6′-O-carbamoyl tobramycin titer starts to decrease and the level of Kanamycin B (contaminant) increase (e.g., the ratio of 6′-O-carbamoyl tobramycin/Kanamycin B is worse). Even if at an aeration rate of 0.2-0.4 vvm, the titer can decrease by 25-50% and the level of Kanamycin B can be doubled. According to the present invention, the impurity formation can be controlled easily using the fed-batch technology using the metabolic controlled fermentation technique. Accordingly, in addition to adjusting the internal pressure and aeration rate of the fermentation, a better demanded optimal value of 6′-O-carbamoyl tobramycin is achieved by continuously feeding assimilable carbon and carbon sources and inorganic phosphate.

Accordingly, the advantages can be effectuated more easily using the continuous feeding relative to the batch-like feeding (See BG 50996 patent).

By the application of a fed-batch process the composition of a simpler initial culture medium can be prepared and it provides a possibility for upgrading it by eliminating the animal originated components (e.g. casein hydrolisate, etc.) and avoiding the potential risk of Bovine Spongiform Encephalopathy (BSE) contamination.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the claims. Various publications are cited herein, the disclosure of which are incorporated by reference in their entireties.

What is claimed is:

1. A process for producing 6′-0-carbamoyl tobramycin from a 6′-0-carbamoyl tobramycin producing microorganism, comprising the steps of:
a) preparing a fermentation broth containing the 6′-0-carbamoyl tobramycin producing microorganism;
b) regulating a constant level of assimilable carbon source and assimilable nitrogen source;
c) recovering the 6′-0-carbamoyl tobramycin.

2. The process of claim 1, wherein the 6′-0-carbamoyl tobramycin producing microorganism is Streptomyces tenebrarius.

3. The process of claim 1, wherein the assimilable carbon source is glucose.

4. The process of claim 3, wherein the glucose is regulated at a constant level in the range of about 0.001 to about 0.5%.

5. The process of claim 3, wherein the glucose is regulated at a constant level in the range of about 0.001 to about 0.4%.

6. The process of claim 3, wherein the glucose is regulated at a constant level in the range of about 0.001 to about 0.05%.

7. The process of claim 1, wherein the assimilable carbon source is glutamic acid.

8. The process of claim 1, wherein the assimilable carbon source is sodium glutamate.

9. The process of claims 7 or 8, wherein the assimilable carbon source is regulated at a constant level in the range of about 0.005 to about 0.1%.

10. The process of claims 7 or 8, wherein the assimilable carbon source is regulated at a constant level in the range of about 0.001 to about 0.1%.

11. The process of claim 1, wherein the assimilable nitrogen source is ammonia nitrogen.

12. The process of claim 11, wherein the ammonia nitrogen is selected from urea, ammonium sulfate, ammonium chloride, ammonium phosphate, ammonium nitrate and the mixtures thereof.

13. The process of claim 11, wherein the ammonia nitrogen is ammonium sulfate.

14. The process of claim 11, wherein the ammonia nitrogen is regulated at a constant level in the range of about 0.03 to about 0.2%.

15. The process of claim 11, wherein the ammonia nitrogen is regulated at a constant level in the range of about 0.02 to about 0.2%.

16. The process of claim 1, wherein a constant level of assimilable carbon source and assimilable nitrogen source in the fermentation broth is regulated by continuously feeding of glucose, sodium glutamate and ammonium sulfate.

17. The process of claim 16, wherein the continuous feeding of glucose, sodium glutamate and ammonium sulfate occur independently of each other.

18. The process of claim 1, further comprising a continuously feeding of a mineral salt.

19. The process of claim 18, wherein the mineral salt is selected from the group consisting of calcium, magnesium, iron, zinc phosphate, manganese, sodium, potassium and cobalt.

20. The process as in claim 4, 5 or 6, wherein the glucose solution is adjusted of a pH between about 4.0 to about 5.0.

21. The process of claim 20, wherein the pH of the glucose solution is adjusted using an inorganic phosphate.

22. The process of claim 21, wherein the inorganic phosphate is phosphoric acid.

23. The process of claim 22, wherein the inorganic phosphate is fed during the fermentation in the quantity of about 0.001 to about 0.002% per day.

24. The process of claim 2, wherein the Streptomyces tenebrarius strain strain is NCAIM B(P) 000169.

25. The process of claim 2, wherein the Streptomyces tenebrarius strain is NCAIM B(P) 000204.

26. The process of claim 1, wherein the fermentation is a submerged culture.

27. The process of claim 1, wherein the fermentation is maintained at a temperature range of about 37 to about 41° C.

28. 6′-0-carbamoyl tobramycin as produced in accordance with the process of claim 1.

29. A formulation useful in treating infectious disease in human comprising 6′-0-carbamoyl tobramycin produced in accordance with the process of claim 1.

30. A formulation useful in treating eye and ear infection in human comprising 6′-0-carbamoyl tobramycin produced in accordance with the process of claim 1.

31. The formulation as in claim 29 or 30, wherein the 6′-0-carbamoyl tobramycin produced in accordance with the process of claim 1 kills bacteria selected from the group consisting of Staphylococcus aureus, Staphylococcs epidermidis, Streptococcus pneumoniae, Psudomonas aeruginosa, Escherichia coli, Enterobacter aerogenes, Proteus mirabelis, Klebsiella pneumoniae, Morganella morgan i, Haemophilus influenzae, Haemophilus aegyptius, Moraxlea lacumata, and Acinetobacter calcoaceticus.
